(19) United States
(12) Patent Application Publication
(10) Pub. No.: US 2025/0084132 A1
(43) Pub. Date: Mar. 13, 2025

(54) T-CELL EPITOPE POLYPEPTIDE OF SENECAVIRUS A AND USES THEREOF

(71) Applicant: Lanzhou Veterinary Research Institute Chinese Academy of Agricultural Sciences, Lanzhou (CN)

(72) Inventors: Huichen GUO, Lanzhou (CN); Suyu MU, Lanzhou (CN); Shiqi SUN, Lanzhou (CN); Shaobin SHANG, Lanzhou (CN); Lingbo CHEN, Lanzhou (CN); Manyuan BAI, Lanzhou (CN); Yun ZHANG, Lanzhou (CN); Jinen WU, Lanzhou (CN); Shuanghui YIN, Lanzhou (CN); Zhidong TENG, Lanzhou (CN); Jingjing ZHOU, Lanzhou (CN); Hu DONG, Lanzhou (CN)

(21) Appl. No.: 18/884,093

(22) Filed: Sep. 12, 2024

(30) Foreign Application Priority Data
Sep. 13, 2023 (CN) 202311180635.X

(51) Int. Cl. C07K 14/005; A61K 39/00

(52) U.S. Cl. CPC C07K 14/005; A61K 39/00; C12N 2770/32022; C12N 2770/32034

(57) ABSTRACT

A T-cell epitope polypeptide of Senecavirus A (SVA) having an amino acid sequence represented by one of SEQ ID NOs: 1-7: SEQ ID NO: 1:

DEALGRVLTPAAVDEALVDL;
SEQ ID NO: 2:
AILAKLGLALAAVTPGLIIL;
SEQ ID NO: 3:
KASPVLQYQL;
SEQ ID NO: 4:
EMKKLGPVAL;
SEQ ID NO: 5:
AHDAFMAGSG;
SEQ ID NO: 6:
PPLGDDQIEYLQVLKSLALT;
and
SEQ ID NO: 7:
LASTLIAQAVSKRLYGSQSV.

### CROSS-REFERENCE TO RELATED APPLICATIONS

Pursuant to 35 U.S.C. § 119 and the Paris Convention Treaty, this application claims foreign priority to Chinese Patent Application No: 202311180635.X filed Sep. 13, 2023, the contents of which, including any intervening amendments thereto, are incorporated herein by reference.

### INCORPORATION-BY-REFERENCE OF SEQUENCE LISTING

This application contains a sequence listing, which has been submitted electronically in XML file and is incorporated herein by reference in its entirety. The XML file, created on Aug. 23, 2024, is named LZSY-00901-UUS.xml, and is 7,012 bytes in size.

### BACKGROUND

The disclosure relates to the field of veterinary biologics, and more particularly, to a T-cell epitope polypeptide of Senecavirus A and uses thereof.

Senecavirus A (SVA) is a main pathogen causing infectious porcine senecavirus disease. The virus has been found to be prevalent only in live pigs, and live pigs are considered to be a natural host of SVA.

SVA is a member of the Picornaviridae family. SVA is a non-enveloped, single-stranded positive-sense RNA virus with an icosahedral structure and a genome length of 7,300 nt, including a 5′-untranslated region (5′-UTR) of 666 nucleotides, a 3′-untranslated region (3′-UTR) of 71 nucleotides, and a unique open reading frame (ORF) between the two non-coding regions.

The ORF of SVA has a typical viral genome structure L-4-3-4 of small RNA, which encodes a polyprotein of 2181 amino acids, where the L region encodes the leading protein L pro; the P1 region encodes four structural proteins: VP1, VP2, VP3, and VP4; the P2 region encodes three nonstructural proteins: 2A, 2B, and 2C, and the P3 region encodes four nonstructural proteins: 3A, 3B, 3C, and 3D. In recent studies, the immunogenicity of structural proteins such as the VP2 protein of SVA has been reported, while the immunogenicity of nonstructural proteins has not been reported.

T cells play an important role in the immune response, and when binding to receptors on the membrane of target cells, cytokines have the ability to destroy tumor cells, inhibit viral replication, and activate macrophages or neutrophils. The role of T cells in controlling SVA infection is not yet fully understood, as SVA has only recently been recognized as an important pathogen infecting swine.

Cellular immunity has an important role in activating the immune response, but there are few vaccines on stimulating the cellular immune response. Studies show immunization with an attenuated vaccine results in the proliferation of memory T-cells (CD4⁺, CD8⁺, and CD4⁺/CD8+ T-cells), demonstrating an effective stimulation of cellular immunity. Conversely, the use of inactivated vaccines, however, does not produce a strong cellular immune response and has a weak protection. Thus, the development of SVA vaccines with cellular immune responses is beneficial for the prevention and control of the disease, but the T-cell epitopes are still unclear and no relevant T-cell epitopes have been reported. Therefore, there is an urgent need to screen peptides that effectively induce cellular immunity and to establish effective methods for evaluating cellular immunity, so as to provide a technological platform for subsequent vaccine research and development and the establishment of evaluation systems.

### SUMMARY

The disclosure screens the T-cell immune antigenic epitopes of two conserved non-structural proteins 2C and 3AB of Senecavirus A (SVA), to obtain a T-cell epitope polypeptide of the non-structural proteins of SVA, which has a high degree of sequence conservatism, and can efficiently induce an SVA-specific immune response.

In one aspect, the first objective of the disclosure is to provide a T-cell epitope polypeptide of Senecavirus A (SVA), the epitope polypeptide having an amino acid sequence represented by one of SEQ ID NOs: 1-7:

SEQ ID NO: 1:
DEALGRVLTPAAVDEALVDL;
SEQ ID NO: 2:
AILAKLGLALAAVTPGLIIL;
SEQ ID NO: 3:
KASPVLQYQL;
SEQ ID NO: 4:
EMKKLGPVAL;
SEQ ID NO: 5:
AHDAFMAGSG;
SEQ ID NO: 6:
PPLGDDQIEYLQVLKSLALT;
and
SEQ ID NO: 7:
LASTLIAQAVSKRLYGSQSV.

In a class of this embodiment, the T-cell epitope polypeptide of Senecavirus A is a polypeptide comprising 80-100% homologous sequences.

In a second aspect, the disclosure provides a construct comprising the nucleic acid molecule encoding the T-cell epitope polypeptide of SVA.

In a third aspect, the disclosure provides a host cell comprising the construct, and/or a cell transformed or transfected by the construct.

In a fourth aspect, the disclosure provides a composition comprising the T-cell epitope polypeptide of SVA and a pharmaceutically acceptable vector or auxiliary.

In a class of this embodiment, the composition further comprises a recombinant protein comprising a T-cell epitope polypeptide of SVA, RNA or DNA of an antigenic polypeptide of SVA, or a vector for expression of the antigenic polypeptide by an attenuated virus or bacterium.

In a class of this embodiment, a fusion protein of the T-cell epitope polypeptide of Senecavirus A has a sequence homology of 80-100%.

In a fifth aspect, the disclosure provides a pharmaceutical composition, comprising the T-cell epitope polypeptide of SVA, the construct, the host cell, or the composition, and a pharmaceutically acceptable auxiliary.

In a sixth aspect, the disclosure provides an antibody, comprising the T-cell epitope polypeptide of SVA.

In a class of this embodiment, the antibody further comprises a nucleic acid molecule of an antigen binding fragment of the antibody.

In a seventh aspect, the disclosure provides an immune composition, comprising the T-cell epitope polypeptide of SVA and an adjuvant.

In a class of this embodiment, the immune composition is in the form of a vaccine, a detection reagent, or a biological diagnostic reagent.

In a class of this embodiment, the vaccine is an amino acid vaccine or a nucleic acid vaccine.

The following advantages are associated with the disclosure. The antigen epitope peptides provided by the disclosure can stimulate strong cellular immune responses and have good immune effects, producing better neutralizing antibodies and complete protection.

### BRIEF DESCRIPTION OF THE DRAWINGS

FIG. 1 shows blood collection for PBMC and sera;

FIG. 2 shows clinical symptoms score of infected pigs;

FIG. 3 shows cellular cytokine staining gating strategy and analysis of IFN-γ secretion;

FIG. 4 shows IFN-γ secretion in porcine PBMC stimulated by a single peptide on days 14 and 28 through ELISpot detection;

FIG. 5 shows flow cytometry gating strategy for Ki67 proliferation assay and analysis of IFN-γ and proliferation effect of CD4 T-cells;

FIG. 6 shows analysis of IFN-γ and proliferation effect of CD8⁺ T-cells;

FIG. 7 shows peptides 2C-5 and 2C-6 can induce the proliferation of CD4/8 T-cells and the secretion of IFN-γ, and 3AB-38 can induce the proliferation of CD4 T-cells and the secretion of IFN-γ;

FIG. 8 shows conservative analysis of T-cell epitopes in 3AB; and

FIG. 9 shows conservative analysis of T-cell epitopes in 2C.

### DETAILED DESCRIPTION

To further illustrate the disclosure, embodiments detailing a T-cell epitope polypeptide of Senecavirus A are described below. It should be noted that the following embodiments are intended to describe and not to limit the disclosure.

The experimental methods in the following examples, unless otherwise stated, are conventional methods. The experimental materials used in the following examples, unless otherwise stated, are commercially available through conventional means.

### Example 1 Design and Synthesis of Overlapping Short Peptides

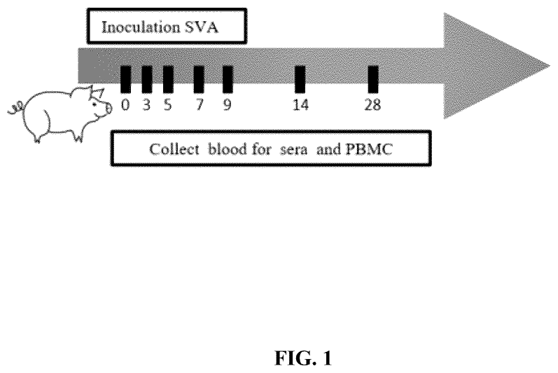

Overlapping peptide libraries of nonstructural proteins 2C and 3AB of SVA (CH-HuB-2017/MN922286) were designed by peptide library design software, and overlapping short peptides with a length of 20 amino acids were synthesized in vitro (see FIG. 1). The samples were infected with SVA at day 0, and blood samples were collected at days 0, 3, 5, 7, 9, 14, and 28 for peripheral blood lymphocytes cells (PBMC) and sera.

### 1. Animal Experiment

### 1.1 Animal Grouping

Animal grouping, virus inoculation method and administration dosage were shown in Table 1. 4 fattening pigs aged 3-4 months old were randomly selected, of which 3 pigs were in the SVA-infected group, and each pig was infected by intramuscular injection (3 mL, 7×10^{7.8} PFU/mL) and nasal drip (3 mL, 7×10^{7.8} PFU/mL) in three parallel experiments, which were numbered as #1; #2; and #3, and the remaining 1 pig was in the control group, numbered #4. No antibodies to SVA were detected in the sera of all animals.

TABLE 1
Animal grouping, virus inoculation method and administration dosage

| | Pig | Inoculation | |
|---|---|---|---|
| Group | ID | Method | Inoculation Dose |
| A: SVA | #1 | Intramuslar + | Intramuscular |
| infect | #2 | Nasal drops | (3 mL, 7 × 10⁷ PFU/mL) and |
| | #3 | | Intranasal |
| | | | (1.5 mL eachnostril, 7 × 1 0^{7.8} PFU/mL) |
| B: Control | #4 | | Intramuscular (3 mL PBS) and |
| (PBS) | | | Intranasal (1.5 mL each nostril PBS) |

### 1.2 Sample Collection

Sample collection was shown in FIG. 1; anticoagulated blood was collected from infected and uninfected pigs on days 0, 3, 5, 7, 9, 14, and 28, respectively, and the collected anticoagulated blood was treated through the Porcine Peripheral Blood Lymphocyte Separation Kit from Solarbio Science & Technology Co., Ltd. for the isolation of PBMCs.

### 1.3 Clinical Symptom Assessment

The clinical symptoms score is shown in Table 2.

TABLE 2
Clinical symptoms score

| Clinical symptoms | Clinical score | Total score |
|---|---|---|
| Left forefoot blisters | 1 | |
| Right forefoot blisters | 1 | |
| Left hindfoot blisters | 1 | 5 |
| Right hindfoot blisters | 1 | |
| Mouth and nose blisters | 1 | |

### 1.4 Viremia Detection

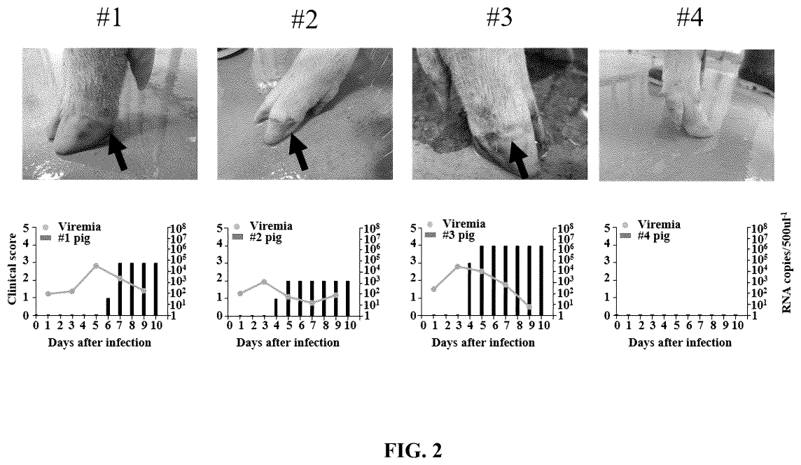

The viral load of the anticoagulated blood was measured by SVA fluorescence quantitative detection kit (refer to Chinese Patent No. CN109652593B), and the results are shown in FIG. 2.

The clinical symptoms and viremia were shown. Pig #1 developed viremia after 4 days' infection and caused blisters on the coronet on the 6th day. Pigs #2 and #3 developed viremia on the 3rd day after infection and caused blisters on the coronet on the 4th day, while the control group showed no symptoms.

### 2. Evaluation of 2C and 3AB T-Cell Responses in Infected Pigs Using Intracellular Staining Techniques

The 2C peptide pool comprising overlapping peptides covering the entire SVA 2C protein was purchased from Nanjing Kingsley Co., Ltd, the 3AB peptide pool comprising overlapping peptides covering the entire SVA 3AB protein was purchased from Nanjing Kingsley Co., Ltd, and the inactivated SVA antigen (iSVA) was obtained by inactivation treatment with binary ethylenimine (BEI) at 30° C. for 28 h. The peptides and inactivated viruses were placed in PBS buffer with a final concentration of 1ug/mL. The peripheral blood lymphocytes were stimulated with 2C, 3AB or iSVA for 12 h in the presence of Brefeldin A (Biolegend).

The cells were first stained using Thermo's LIVE/DEAD Violet for 30 min to differentiate between live and dead cells, then stained in 1% FCS/PBS buffer containing BD's anti-porcine CD3, CD4, CD8a, γδ T antibodies for 15 min, washed for 15 min, and fixed for 15 min in BD Cell Membrane/Cell Treatment Solution. Finally, intracellular staining was performed in PERM/WASH buffer using BD's anti-porcine IFN-G or anti-human TNF antibody for 20 min at 4° C. and detected by BD FACS flow cytometer.

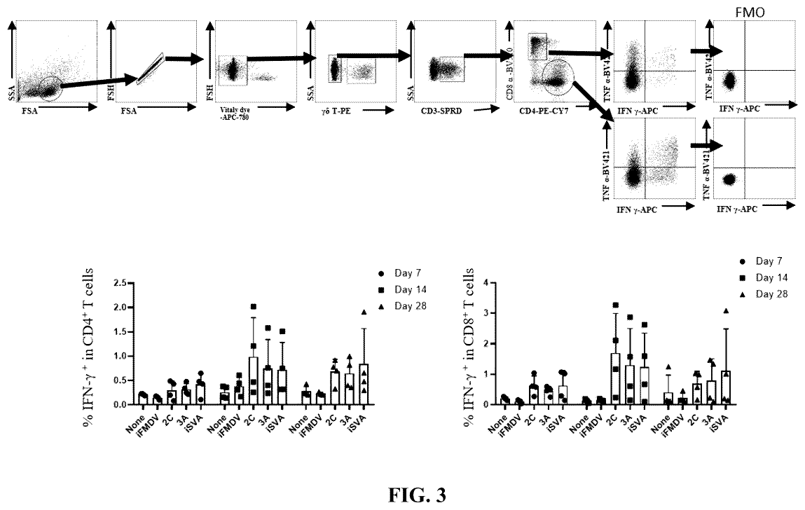

As shown in FIG. 3, the kinetics of CD4⁺ and CD8⁺ T cell responses in the circulation after SVA infection were detected. PBMCs were stimulated with purified and inactivated SVA, inactivated FMDV, and 2C and 3A peptide libraries, and the expression of IFN-γ in the CD4⁺ and CD8⁺ T cells was analyzed by intracellular staining. Representative data were observed on the 7th, 14th, and 35th days after SVA infection.

The results showed that the secretion of IFN-γ was detected in pigs #1, #2, #3, and #4 on days 7, 14, and 28, respectively. On the 14^{th} day, CD4⁺ T and CD8⁺ T cells produced the most IFN-γ.

### Example 2 Screening of T-Cell Epitopes

### 1. Identification of T-Cell Epitopes Through ELISPOT

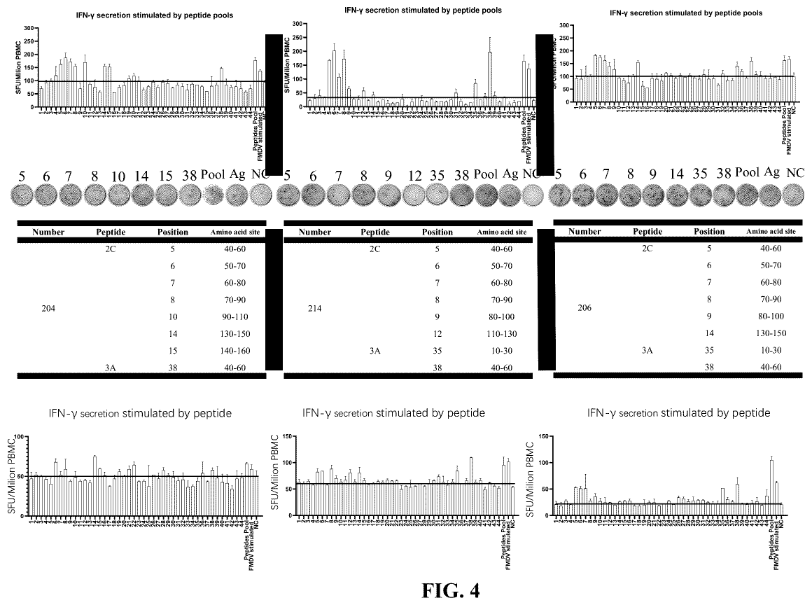

Anticoagulated blood was collected from SVA-infected pigs at 14 and 28 days, lymphocytes were isolated and stimulated with the abovementioned single peptides and peptide pools, and the IFN-γ secretion in porcine peripheral blood lymphocytes cells (PBMC) was detected using a commercial IFN-γ ELISPOT kit (Mabtech, Nacka Strand, Sweeden), and counted by spot-forming units. As shown in FIG. 4, a strong T-cell response was detected by IFN-γ ELISPOT on the 14th day after SVA infection, and positively reactive peptides were detected from 44 single peptide stimuli from 2C and 3AB. Specifically, single peptides 5, 6, 7, 8, 9, 12, 35, and 38 were found to be reactive with IFN-γ.

The synthesized single 2C and 3AB peptides were screened, where the vertical coordinate unit of the graph is the number of spots per 5×10⁶ cells. Each peptide of the 3AB and 2C peptide libraries contained 20 amino acids, and adjacent peptides contained 10 overlapping amino acid sequences. 44 peptides were synthesized in total, which were named as 1-44. Porcine PBMCs were stimulated with each of the peptides of 3AB and 2C peptide libraries respectively, and the number of spots formed by ELISPOT coloring was counted. The dominant epitope peptides were screened twice, and a total of 11 peptides were obtained, which were 2C-5, 6, 7, 8, 9, 10, 12, 14, 15; 3AB-35, 38, of which the results were consistent on the 14th day and the 28th day.

### 2. Phenotyping and Identification of T-Cell Epitopes

Cell surface and intracellular cytokine staining was used to detect T-cell subpopulation reactivity of peptides 2C-5, 6, 7, 8, 9, 12 and 3AB-35, 38 obtained by ELISpot detection. PBMC cells were washed with 1640 medium and co-incubated with the monopeptides, the ionophore transport inhibitors GolgiStop and GolgiPlug in 96-well culture plates for 12 hours at 37° C. Live and dead cells were first differentiated using LIVE/DEAD Violet staining, followed by surface staining of PBMC cells using anti-porcine CD3⁺, CD4⁺, CD8⁺ and γδ T-cell antibodies to differentiate different lymphocyte subpopulations. Finally, intracellular cytokine staining was performed with anti-porcine IFN-γ, TNF-α and other antibodies according to the instructions using the BD Cytofix/Cytoperm solution (BD Biosciences) kit, and the results are shown in Table 3.

Cell surface and intracellular cytokine staining was used to detect T-cell subpopulation reactivity of peptides 2C-5, 6, 7, 8, 9, 12 and 3AB-35, 38 obtained by ELISpot detection. The PBMC cells were washed with 1640 medium and co-incubated with the monopeptides in 96-well culture plates for 72 hours at 37° C.

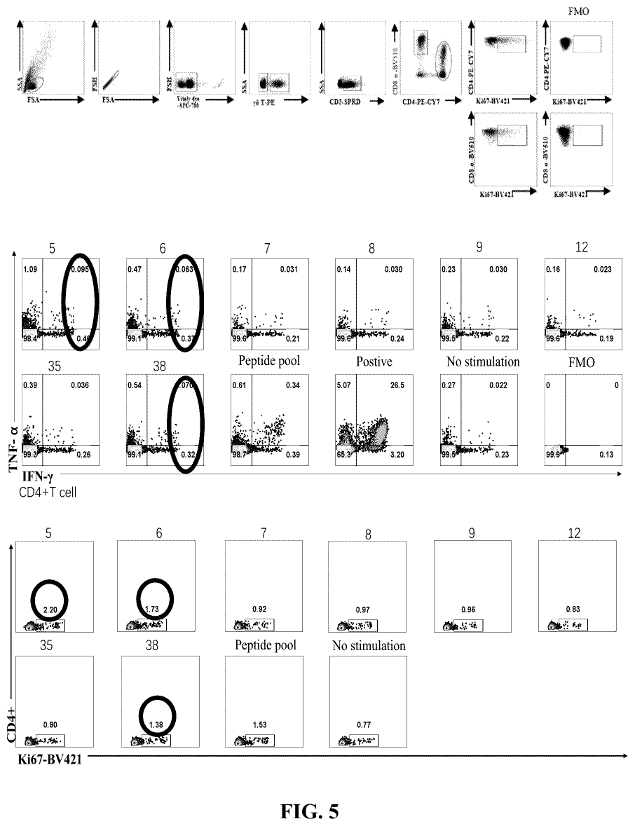

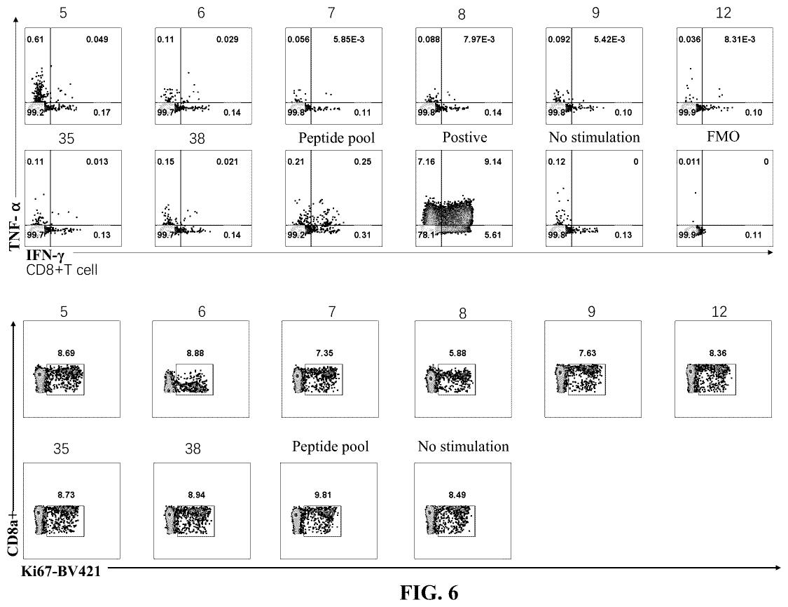

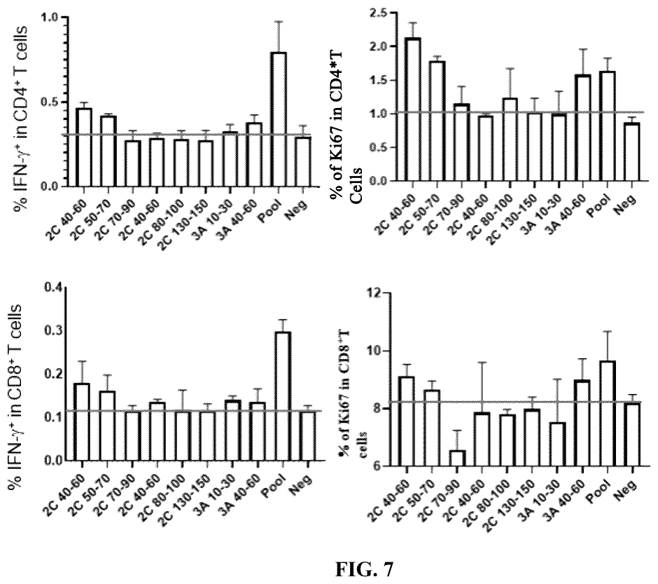

The cells were first stained using Thermo's LIVE/DEAD Violet for 30 min to differentiate between live and dead cells, then stained in 1% FCS/PBS buffer containing BD's anti-porcine CD3, CD4⁺, CD8⁺a, γδ T antibodies for 15 min, washed for 15 min, and fixed for 15 min in BD Cell Membrane/Cell Treatment Solution. Finally, intracellular staining was performed in PERM/WASH buffer using BD's Ki-67 antibody for 20 min at 4° C. and detected by BD FACS flow cytometer. The results are shown in FIGS. 5-7. As shown in FIGS. 5-7, the cells were stained with intracellular, nuclear, and extracellular markers and analyzed by FACS. Pigs with 14-days' infection were selected and PBMC cells were isolated and stimulated with peptides selected by ELISpot. The MHC-I and MHC-II restriction of the identified peptide segments were determined through intracellular factor staining (IFN-γ) and nuclear staining (Ki67).

The results showed that after stimulation of CD4⁺ and CD8⁺ T-cells with T-cell epitope peptides, Ki67 staining assay was performed to assess the proliferation and cellular immune activation of CD4⁺ and CD8⁺ T-cells. The results showed that the highly reactive peptides identified in ELISPOT assay can induce a significant increase in the proliferation rate of CD4⁺ T-cells in porcine PBMCs infected with SVA.

TABLE 3
Intracellular cytokine staining of porcine antibodies IFN-γ and TNF-α

| | | | | T-cell |
|---|---|---|---|---|
| No. | peptide | Position | Amino acid site | identification |
| 214 | 2C | 5 | 40-60 | CD4/CD8 |
| | | 6 | 50-70 | CD4/CD8 |
| | | 7 | 60-80 | / |
| | | 8 | 70-90 | / |
| | | 9 | 80-100 | / |
| | | 12 | 110-130 | / |
| | 3AB | 35 | 10-30 | / |
| | | 38 | 40-60 | CD4 |

### 3. Conservative Analysis of T-Cell Epitopes

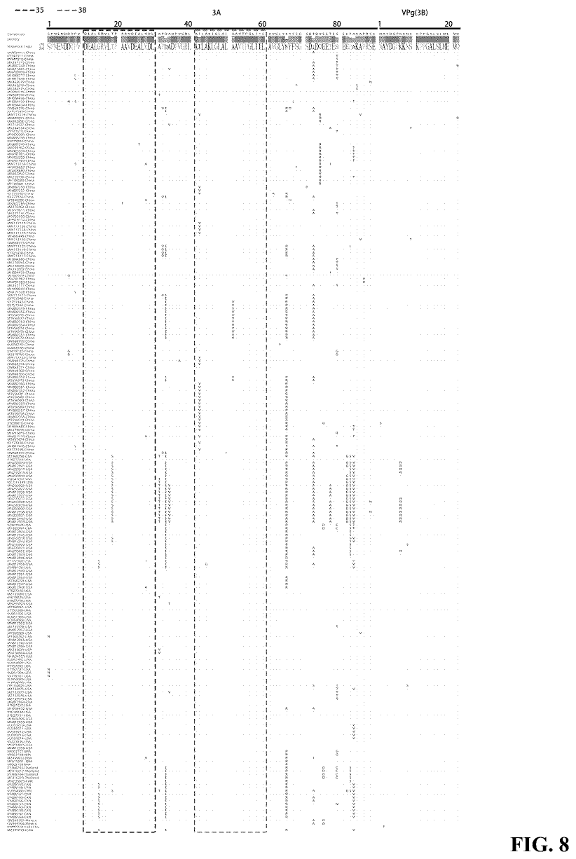

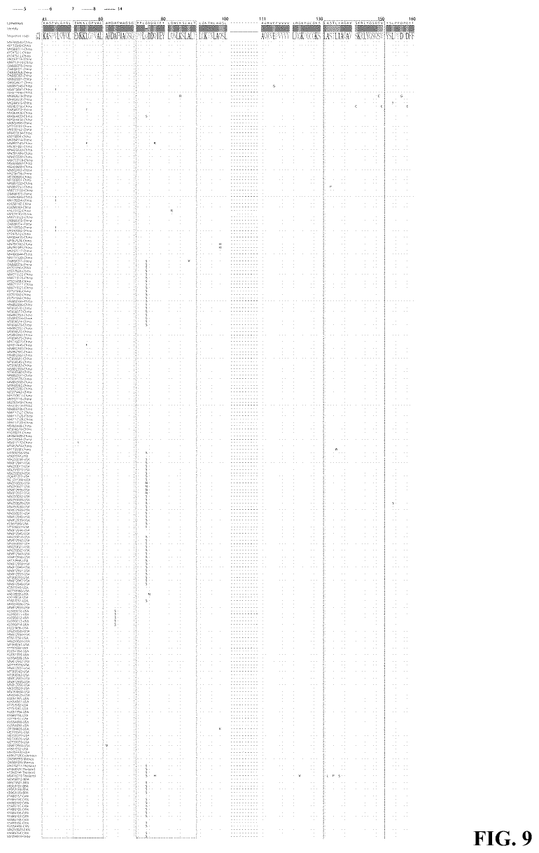

To analyze the conservation of the screened SVA 2C and 3AB T-cell epitopes, the 237 SVA amino acid sequences currently available in the NCBI database were downloaded and aligned using Geneious Prime software to determine the number of virulent strains recognized by the epitopes as well as the conservation analysis (shown in FIGS. 8 and 9), and the sequence list is shown in Table 4.

TABLE 4
Amino acid sequence

| Non-structural | | |
|---|---|---|
| protein | Sequence name | Amino acid sequence |
| 3AB | SEQ ID NO: 1 | DEALGRVLTPAAVDEALVDL |
| | SEQ ID NO: 2 | AILAKLGLALAAVTPGLIIL |
| 2C | SEQ ID NO: 3 | KASPVLQYQL |
| | SEQ ID NO: 4 | EMKKLGPVAL |
| | SEQ ID NO: 5 | AHDAFMAGSG |
| | SEQ ID NO: 6 | PPLGDDQIEYLQVLKSLALT |
| | SEQ ID NO: 7 | LASTLIAQAVSKRLYGSQSV |

### Example 3 Construction and Immune Efficacy Evaluation of SVA T/B Vaccine

The experimental pigs were divided into four groups, one group immunized with VLP vaccine alone, one group immunized with T-cell epitope+VLPs vaccine, one group immunized with T-cell epitope vaccine alone, and one group not immunized as a negative control. The initial and booster immunization strategies were used, and the virus was attacked 7 days after the booster immunization to observe the onset of the disease and detect the viral loads of blood, oral cavity, nasal swabs, intestinal swabs, and important immune organs of the pigs. The T-cell epitope+VLPs vaccine group was found to produce better neutralizing antibodies than the VLPs or T-cell epitope groups alone and produced complete protection, see Table 5.

TABLE 5
Auxiliary role of T-cell epitopes in vaccines

| Experimental group | No. | 14 day | 21 day | 28 day |
|---|---|---|---|---|
| 201 + VLPs + T-cell | 1 | 1:8 | 1:256 | 1:1024 |
| epitopes | 2 | 1:8 | 1:256 | 1:1024 |
| | 3 | 1:8 | 1:256 | 1:1024 |
| | 4 | 1:8 | 1:128 | 1:512 |
| | 5 | 1:8 | 1:256 | 1:1024 |
| | 6 | 1:8 | 1:512 | 1:1024 |
| 201 + VLPs | 7 | <1:4 | 1:128 | 1:256 |
| | 8 | <1:4 | 1:128 | 1:256 |
| | 9 | <1:4 | 1:64 | 1:512 |
| | 10 | <1:4 | 1:128 | 1:256 |
| | 11 | <1:4 | 1:128 | 1:256 |
| 201 + T-cell epitopes | 12 | <1:4 | <1:4 | <1:4 |
| | 13 | <1:4 | <1:4 | <1:4 |
| | 14 | <1:4 | <1:4 | <1:4 |
| | 15 | <1:4 | <1:4 | <1:4 |
| | 16 | <1:4 | <1:4 | <1:4 |
| Ctrl | 17 | <1:4 | <1:4 | <1:4 |
| | 18 | <1:4 | <1:4 | <1:4 |
| | 19 | <1:4 | <1:4 | <1:4 |

It will be obvious to those skilled in the art that changes and modifications may be made, and therefore, the aim in the appended claims is to cover all such changes and modifications.

What is claimed is:

1. A T-cell epitope polypeptide of Senecavirus A (SVA), the epitope polypeptide having an amino acid sequence represented by one of SEQ ID NOs: 1-7:
SEQ ID NO: 1:
DEALGRVLTPAAVDEALVDL;
SEQ ID NO: 2:
AILAKLGLALAAVTPGLIIL;
SEQ ID NO: 3:
KASPVLQYQL;
SEQ ID NO: 4:
EMKKLGPVAL;
SEQ ID NO: 5:
AHDAFMAGSG;
SEQ ID NO: 6:
PPLGDDQIEYLQVLKSLALT;
and
SEQ ID NO: 7:
LASTLIAQAVSKRLYGSQSV.

2. A construct, comprising a nucleic acid molecule encoding the T-cell epitope polypeptide of SVA of claim 1.

3. A host cell, comprising the construct of claim 2, and/or a cell transformed or transfected by the construct.

4. A composition, comprising the T-cell epitope polypeptide of SVA of claim 1 and a pharmaceutically acceptable vector or auxiliary.

5. The composition of claim 4, further comprising a recombinant protein comprising the T-cell epitope polypeptide of SVA, RNA or DNA of an antigenic polypeptide of SVA, or a vector for expression of the antigenic polypeptide by an attenuated virus or bacterium.

6. A pharmaceutical composition, comprising the T-cell epitope polypeptide of SVA of claim 1, a construct comprising the T-cell epitope polypeptide of SVA, a host cell comprising the construct, or a composition comprising the T-cell epitope polypeptide of SVA, and a pharmaceutically acceptable auxiliary.

7. An antibody, comprising the T-cell epitope polypeptide of SVA of claim 1.

8. The antibody of claim 7, further comprising a nucleic acid molecule of an antigen binding fragment of the antibody.

9. An immune composition, comprising the T-cell epitope polypeptide of SVA of claim 1 and an adjuvant.

10. The immune composition of claim 9, being in the form of a vaccine, a detection reagent, or a biological diagnostic reagent.


SEQUENCE LISTING (7 sequences)

SEQ ID NO: 1 - protein, 20 aa, synthetic construct
DEALGRVLTPAAVDEALVDL

SEQ ID NO: 2 - protein, 20 aa, synthetic construct
AILAKLGLALAAVTPGLIIL

SEQ ID NO: 3 - protein, 10 aa, synthetic construct
KASPVLQYQL

SEQ ID NO: 4 - protein, 10 aa, synthetic construct
EMKKLGPVAL

SEQ ID NO: 5 - protein, 10 aa, synthetic construct
AHDAFMAGSG

SEQ ID NO: 6 - protein, 20 aa, synthetic construct
PPLGDDQIEYLQVLKSLALT

SEQ ID NO: 7 - protein, 20 aa, synthetic construct
LASTLIAQAVSKRLYGSQSV